(19) United States
(12) Patent Application Publication
(10) Pub. No.: US 2004/0232042 A1
(43) Pub. Date: Nov. 25, 2004

(54) Amine-acid reaction products as asphaltene dispersants in crude oil

(76) Inventor: Ravindranath Mukkamala, Lansdale, PA (US)

Correspondence Address: ROHM AND HAAS COMPANY, PATENT DEPARTMENT, 100 INDEPENDENCE MALL WEST, PHILADELPHIA, PA, 19106-2399, US

(21) Appl. No.: 10/444,160

(22) Filed: May 23, 2003

(51) Int. Cl. C07C 7/20; C10G 9/16

(52) U.S. Cl. 208/48 R; 208/48 AA

(57) ABSTRACT

A composition useful for dispersing asphaltenes in crude oil comprising: (a) 0.001% to 0.2% of at least one reaction product of: (i) an amine; and (ii) a carboxylic, phosphonic or sulfonic acid; and (b) crude oil.

### BACKGROUND

This invention relates generally to a method or dispersing asphaltenes in processing of crude oil.

Certain petroleum products, including heavy crude oils, which include materials referred to as “tars,” “petroleum tars” or “tar sands,” are rich in asphaltenes, metals and resins. The presence of these types of compounds can lead to various problems in the recovery, transportation, treatment and refining of crude oils, including increased viscosity, formation of stable emulsions, fouling and corrosion. U.S. patent application Ser. No. 09/514,462 discloses the use of amine-chelate complexes to reduce the viscosity of crude oil. However, this reference does not disclose a method for dispersing asphaltenes.

The problem addressed by this invention is to find materials suitable for improving processing of petroleum products.

### STATEMENT OF INVENTION

This invention is directed to a composition comprising: (a) 0.001% to 5% of at least one reaction product of: (i) an amine; and (ii) a carboxylic, phosphonic or sulfonic acid; and (b) crude oil; provided that the reaction product has a hydrogen bond donor group located two to eight chemical bonds from either: (i) a carbonyl carbon of a carboxylic acid, a phosphorus atom of a phosphonic acid or a sulfur atom of a sulfonic acid; or (ii) a nitrogen atom of an amino group.

This invention is directed further to a method for dispersing asphaltenes in a petroleum product by adding to the petroleum product 0.001% to 5% of at least one reaction product of: (i) an amine; and (ii) a carboxylic, phosphonic or sulfonic acid; and (b) crude oil; provided that the reaction product has a hydrogen bond donor group located two to eight chemical bonds from either: (i) a carbonyl carbon of a carboxylic acid, a phosphorus atom of a phosphonic acid or a sulfur atom of a sulfonic acid; or (ii) a nitrogen atom of an amino group.

### DETAILED DESCRIPTION

All percentages are weight percentages based on the entire composition, unless otherwise indicated. A “hydrogen bond donor group” is a group capable of forming a hydrogen bond with an oxygen or nitrogen atom, e.g., a hydroxy, oxime, amide, or alkyl amide functional group. An “alkyl” group is a hydrocarbyl group having from one to twenty-two carbon atoms in a linear, branched or cyclic arrangement. Alkyl groups optionally have one or more double or triple bonds. Substitution on alkyl groups of one or more of halo, cyano, alkyl, alkoxy, or the aforementioned polar groups is permitted; alkoxy groups may in turn be substituted by one or more halo substituents. A “heteroalkyl” group is an alkyl group in which at least one carbon has been replaced by O, NR, or S, wherein R is hydrogen, alkyl, heteroalkyl, aryl or aralkyl. An “aryl” group is a substituent derived from an aromatic hydrocarbon compound. An aryl group has a total of from six to twenty ring atoms, and has one or more rings which are separate or fused. An “aralkyl” group is an “alkyl” group substituted by an “aryl” group. A “heterocyclic” group is a substituent derived from a heterocyclic compound having from five to twenty ring atoms, at least one of which is nitrogen, oxygen or sulfur. Preferably, heterocyclic groups do not contain sulfur. Substitution on aryl or heterocyclic groups of one or more of halo, cyano, alkyl, heteroalkyl, alkoxy or the aforementioned polar groups is permitted, with substitution by one or more halo groups being possible on alkyl, heteroalkyl or alkoxy groups. An “aromatic heterocyclic” group is a heterocyclic group derived from an aromatic heterocyclic compound. Preferably, heterocyclic groups in compounds used in this invention are aromatic heterocyclic groups.

An “organic functional group” is a functional group which does not contain metal atoms, and which has from one to twenty-two carbon atoms, hydrogen atoms, and optionally, contains heteroatoms, including but not limited to: nitrogen, oxygen, sulfur, phosphorus and halogen atoms. An organic functional group optionally contains double and/or triple bonds; rings, which are linked or fused; and if it is wholly or partly acyclic, the acyclic part can be linear or branched. Preferably, an organic functional group is an alkyl, heteroalkyl, aryl, aralkyl, heterocyclic or heterocyclic-alkyl group. In a preferred embodiment of this invention, at least one of the organic functional groups is a C₂-C₂₂ alkyl or heteroalkyl group, more preferably a C₇-C₂₂ alkyl or heteroalkyl group, more preferably a C₉-C₂₂ alkyl or heteroalkyl group, and most preferably, a C₁₅-C₂₂ alkyl group.

In the present invention, at least one reaction product of an amine and an acid is added to a petroleum product, with the total amount of said reaction product(s) being from 0.001% to 5%, preferably from 0.01% to 5%, more preferably from 0.01% to 1%, more preferably from 0.01% to 0.2%, and most preferably from 0.02% to 0.2%. The reaction product has no new covalent bonds, i.e., bonds not present in the amine or the acid. The reaction product is either a salt or a physical mixture or complex of the amine and the acid. Preferably, a reaction product used in this invention is a salt, preferably one that is soluble in oil at least at the aforementioned levels. Preferably, the salt has at least ten carbon atoms, more preferably at least 15 carbon atoms. Preferably a salt used in this invention has a cation and an anion, and is not zwitterionic.

In a preferred embodiment of this invention, the acid is a carboxylic acid having no other acidic functional groups, i.e., groups having pKₐ<6. In another preferred embodiment of this invention, the acid is a phosphonic acid having no other acidic functional groups. In another preferred embodiment of this invention, the acid is a sulfonic acid having no other acidic functional groups.

The separation between a hydrogen bond donor group and the carboxylic, phosphonic or sulfonic acid or the amine is measured by the number of covalent chemical bonds intervening between either: (i) an oxygen of a hydroxy group; or (ii) a carbonyl or imine carbon of amide or oxime; and one of: the carboxylic acid carbonyl carbon, the phosphorus atom of a phosphonic acid, the sulfur atom of a sulfonate and the amine nitrogen atom. For example, in an amine salt of glycolic acid (hydroxyacetic acid), the oxygen of the hydroxy group is two bonds from the carbonyl carbon of the carboxylate group. Preferably, at least one polar group in a compound of this invention is located two to six chemical bonds from a carboxylic acid carbonyl carbon, the phosphorus atom of a phosphonic acid, the sulfur atom of a sulfonate or the amine nitrogen atom; and most preferably two, three, four or five chemical bonds from a carboxylic acid carbonyl carbon, the phosphorus atom of a phosphonic acid, the sulfur atom of a sulfonate or the amine nitrogen atom.

A compound used in this invention optionally contains other carboxylate, phosphonate, sulfonate or protonated imine functional groups on one or more of the organic functional groups, preferably for a total of one to five conjugate acid functional groups and one to five conjugate base functional groups. Preferably, the compound has one conjugate acid group and one conjugate base group. Preferably, a hydrogen bond donor group is a hydroxy or amide functional group. Most preferably, a hydrogen bond donor group is a hydroxy group.

In a preferred embodiment of the invention in which a salt of an amine and a carboxylic acid is added to crude oil, the salt has formula (I),

R¹R²R³N+ R⁴COO— (I)

wherein R¹, R², R³ independently are hydrogen or organic functional groups, provided that at least one is not hydrogen; and R⁴ is an organic functional group. Other carboxylate groups or other protonated amine groups optionally are present on R¹, R², R³ or R⁴. Preferably, R¹, R², R³ and R⁴ independently are hydrogen, alkyl, heteroalkyl, aryl, aralkyl or heterocyclic. Preferably, R¹, R², R³ and R⁴ do not contain other carboxylate groups. In one preferred embodiment, one of R¹, R² and R³ is C₁₀-C₂₂ alkyl, more preferably C₁₂-C₂₂ alkyl, and most preferably, C₁₆-C₂₂ alkyl. In one preferred embodiment, the protonated amine part of the compound of formula (I) is derived from an unsubstituted C₁₀-C₂₂ alkyl amine, R²NH₂, preferably one which is an oil-soluble amine. In one embodiment, the alkyl amine is a tertiary alkyl primary amine, i.e., a primary amine in which the alkyl group is attached to the amino group through a tertiary carbon. Examples of commercially available tertiary alkyl primary amines are the Primene™ amines available from Rohm and Haas Company, Philadelphia, Pa. Preferably, a polar group is present and is located two to ten chemical bonds from either: a carbonyl carbon of a carboxylate group; or a nitrogen atom of a protonated imine group.

In one preferred embodiment of the invention, the carboxylate ion of a salt used in this invention has formula (II)

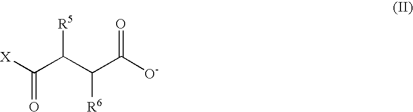

wherein R⁵ and R⁶ independently are hydrogen, alkyl or hydroxy; X is O⁻ or R⁷R⁸N; and R⁷ and R⁸ independently are hydrogen, alkyl, aryl, aralkyl or heteroalkyl. Preferably, R⁷ is hydrogen and R⁸ is alkyl. In one preferred embodiment, R⁵ and R⁶ both are hydroxy and X is O⁻. In another preferred embodiment, R⁵ is alkyl, X is R⁷R⁸N, and R⁶ is hydrogen; preferably, at least one of R⁵, R⁷ and R⁸ is C₈-C₂₂ alkyl, more preferably C₁₆-C₂₂ alkyl. The carboxylate ion of formula (II) occurs in a salt with at least one protonated amine. Preferably, the amine is an unsubstituted alkyl amine, preferably a C₁₆-C₂₂ alkyl amine.

In a preferred embodiment of this invention, the carboxylate ion of a salt is selected from the group consisting of:

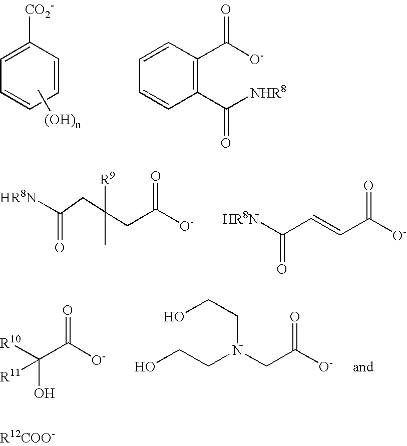

wherein R⁹ is hydrogen or C₁-C₂₂ alkyl; R¹⁰ and R¹¹ independently are hydrogen or C₁-C₂₂ alkyl; R¹² is C₁₆-C₂₂ alkyl; and n is one or two. Preferably, R⁹ is hydrogen or C₁-C₆ alkyl. Preferably, R¹⁰ and R¹¹ independently are hydrogen or C₁-C₆ alkyl, most preferably hydrogen, methyl or ethyl.

In a preferred embodiment of the invention, the ammonium ion of a salt used in the invention has the formula R¹³R¹⁴R¹⁵NH+, wherein R¹³ is C₂-C₆ alkyl, or C₂-C₆ heteroalkyl with one or two carbon atoms replaced by oxygen atoms, and in either case substituted by one or two hydroxy groups; and R¹⁴ and R¹⁵ independently are hydrogen, methyl or ethyl. Preferably, R¹³ is 2-hydroxyethyl or 3-hydroxypropyl. Preferably, R¹⁴ and R¹⁵ are hydrogen. Preferably, the carboxylate ion of the salt is selected from the group consisting of:

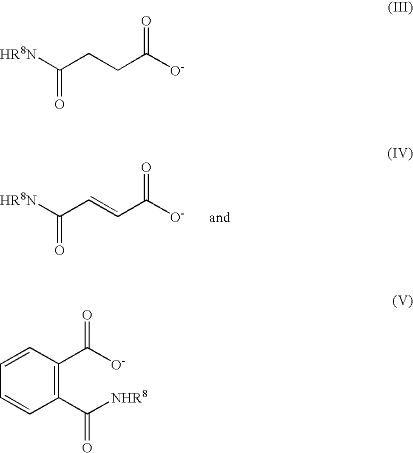

In addition to dispersing asphaltenes, the composition of the present invention typically also increases demulsibility, reduces sediment formation, reduces surface fouling and reduces corrosion. For crude oil recovery, the composition of the present invention can be injected directly into an injection well, or preferably diluted with solvent prior to injection. Suitable solvents include but are not limited to: petroleum distillates such as kerosene and gas oil; linear and branched aliphatic solvents such as pentane, hexanes, mixtures of nonanes and 2-ethylhexanes; cycloaliphatic mixtures commonly known as naphtha; aromatic solvents such as toluene, xylenes and commercial aromatic solvent mixtures; esters; ethers; alcohols such as ethanol, isopropanol, octanol and dodecanol; ketones such as acetone, cyclohexanone and acetophenone; and other polar solvents. Preferred dilutions are 0.01 to 50 wt % of the compound in the solvent, more preferred dilutions being 0.01 to 20 wt %, more preferred dilutions being 0.1 to 10%, and most preferred dilutions being 1 to 10 wt %.

### EXAMPLES

Performance Criteria and Test Methods:

Asphaltene Dispersancy-test tube method: This test requires a previously made dispersion of asphaltene in xylenes (Aromatic 150 solvent) or asphaltenic heavy crude diluted in xylenes (Aromatic 150 solvent) at a known concentration. A solution of an additive formulation (0.1 mL, the active ingredient was typically at 5-10 wt %, making the treat rate 500-1000 ppm) was taken in to a 15.0 mL graduated glass centrifuge tube, and hexanes added such that the total volume in the tube became 10.0 mL. To this mixture of additive and hexanes, asphaltenic stock solution (0.1 mL) was added. The test tube was then capped, shaken vigorously for about a minute or 40-60 times by hand and allowed to stand. The volume of any precipitated asphaltenes settled at the bottom of the tube was recorded at 10, 30, 60, 90 and 1440 (24 h) min intervals. When no additive was used, the volume of asphaltenes precipitated in the first 0.5-1 h was 0.4-0.5 mL (4-5%); in fact, it was important to initially adjust the concentration of the asphaltene stock in such a way that under these conditions of dilution with paraffinic solvents, a 4-5 vol % of asphaltenic precipitation occurred. When the additive was an effective dispersant of asphaltene, then no precipitate was formed up to 24 h (Rating=2; good). In some cases, no precipitation was observed in over 24 h to several days (Rating=2+; excellent). If the additive was not a dispersant, then an almost immediate precipitation of asphaltenes occurred (Rating=0; poor). Results for several amine-carboxylic acid salts are reported in the Table.

TABLE
Salts of hydroxy acids and amines

| | | |
|---|---|---|
| 1 | salicylic acid/oleylamine (1:1) salt | 2+ |
| | hexanes; treat rate: 1000 ppm | |
| 2 | oleylamine/2-hydroxyisobutyric acid salt | 2+ |
| | hexanes; treat rate: 1000 ppm | |
| 3 | oleylamine/3,4-dihydroxybenzoic acid salt | 2+ |
| | hexanes; treat rate: 1000 ppm | |
| 4 | oleylamine/bicine salt | 2+ |
| | hexanes; treat rate: 1000 ppm | |
| 5 | oleylamine/citric acid; 2:1 salt | 2+ |
| | hexanes; treat rate: 1000 ppm | |
| 6 | oleylamine/citric acid; 3:1 salt | 2+ |
| | hexanes; treat rate: 1000 ppm | |

Salts of hydroxy amines and acids

| | | |
|---|---|---|
| 7 | Sylfat ™ FA-1/ethanolamine salt | 2+ |
| | hexanes; 1000 ppm | |
| 8 | Sylfat ™ FA-1/diethanolamine salt | 1 |
| | hexanes; 1000 ppm | |
| 9 | N-Methy1-N-oleyoylglycine/ethanolamine salt | 2 |
| | hexanes; 1000 ppm | |
| 10 | ethanolamine/III, R⁸ = C-16-22 (Primene ™ JM-T) salt | |
| | hexanes; 2000 ppm | 2+ |
| | Aromatic 150; 1000 ppm | 2+ |
| 11 | ethanolamine/IV, R⁸ = C-16-22 (Primene ™ JM-T) salt | 2+ |
| | hexanes; 2000 ppm | |
| 12 | ethanolamine/V, R⁸ = C-16-22 (Primene ™ JM-T) salt | 2+ |
| | hexanes; 2000 ppm | |

The treat rate is the concentration of the salt in the crude oil. AROMATIC 150 SOLVENT is a solvent mixture with a boiling range of 184-204° C. which contains xylene isomers, and which is available from Exxon Mobil Chemical Co., Houston Tex.; this solvent, at a treat rate of 10,000 ppm has a rating of 0.

Primene™ amines are tertiary alkyl primary amines in which the alkyl groups are a mixture of isomers. In Primene™ 81-R amine, the alkyl group is a mixture of C10-C₁₅ alkyl groups; and in Primene™ JM-T amine, of C₁₆-C₂₂ alkyl groups.

Sylfat™ FA-1 is a naturally-occurring mixture of unsaturated C₁₈ aliphatic acids, available from Arizona Chemicals Co., Jacksonville, Fla.

Efficacy of viscosity reduction in crude oil was assessed for salts 1-6 (1 and 2% treat rates), 7 (2000 ppm) and 10 (4000 ppm). None of these salts reduced the viscosity of the oil.

1. A composition comprising:
(a) 0.001% to 0.2% of at least one reaction product of: (i) an amine; and (ii) a carboxylic, phosphonic or sulfonic acid; and
(b) crude oil;
provided that the reaction product has a hydrogen bond donor group located two to eight chemical bonds from either: (i) a carbonyl carbon of a carboxylic acid, a phosphorus atom of a phosphonic acid or a sulfur atom of a sulfonic acid; or (ii) a nitrogen atom of an amino group.

2. The composition of claim 1 in which said reaction product is a salt of a carboxylic, phosphonic or sulfonic acid, and the salt has a polar group located two to six chemical bonds from either: (i) a carbonyl carbon of a carboxylate group, a phosphorus atom of a phosphonate group or a sulfur atom of a sulfonate group; or (ii) a nitrogen atom of a protonated amine.

3. The composition of claim 2 in which said hydrogen bond donor group is hydroxy or oxime.

4. The composition of claim 3 in which said hydrogen bond donor group is hydroxy, and is located two to five chemical bonds from either: (i) a carbonyl carbon of a carboxylate group, a phosphorus atom of a phosphonate group or a sulfur atom of a sulfonate group; or (ii) a nitrogen atom of a protonated amine.

5. The composition of claim 4 in which said at least one salt is a salt of an amine and a carboxylic acid, and has no other acidic functional groups.

6. A method for dispersing asphaltenes in a petroleum product; said method comprising adding to the petroleum product 0.001% to 0.2% of at least one reaction product of: (i) an amine; and (ii) a carboxylic, phosphonic or sulfonic acid;
provided that the reaction product has a hydrogen bond donor group located two to eight chemical bonds from either: (i) a carbonyl carbon of a carboxylic acid, a phosphorus atom of a phosphonic acid or a sulfur atom of a sulfonic acid; or (ii) a nitrogen atom of an amino group.

7. The method of claim 6 in which said reaction product is a salt of a carboxylic, phosphonic or sulfonic acid, and the salt has a polar group located two to six chemical bonds from either: (i) a carbonyl carbon of a carboxylate group, a phosphorus atom of a phosphonate group or a sulfur atom of a sulfonate group; or (ii) a nitrogen atom of a protonated amine.

8. The method of claim 7 in which said hydrogen bond donor group is hydroxy or oxime.

9. The method of claim 8 in which said hydrogen bond donor group is hydroxy, and is located two to five chemical bonds from either: (i) a carbonyl carbon of a carboxylate group, a phosphorus atom of a phosphonate group or a sulfur atom of a sulfonate group; or (ii) a nitrogen atom of a protonated amine.

10. The method of claim 9 in which said at least one salt is a salt of an amine and a carboxylic acid, and has no other acidic functional groups.
